(19) United States
(12) Patent Application Publication
(10) Pub. No.: US 2025/0009528 A1
(43) Pub. Date: Jan. 9, 2025

(54) HYDRAULIC ACTUATOR AND CONTROL VALVE

(71) Applicant: OTTO BOCK HEALTHCARE PRODUCTS GMBH, Wien (AT)

(72) Inventor: Zsolt Tamas SZTRANCSIK, Wien (AT)

(21) Appl. No.: 18/739,484

(22) Filed: Jun. 11, 2024

(30) Foreign Application Priority Data
Jul. 4, 2023 (DE) 10 2023 117 663.3

(51) Int. Cl. A61F 2/60; A61F 2/50; A61F 2/74; F16K 1/12

(52) U.S. Cl. CPC A61F 2/60; A61F 2/748; F16K 1/126; A61F 2002/5038

(57) ABSTRACT

A hydraulic actuator for orthopaedic devices has a control valve in a flow channel which has an inlet and an outlet for a hydraulic fluid. The control valve has a valve sleeve and a valve member. The valve sleeve has a sleeve wall which forms a receiving space and in which at least one radially orientated inlet opening is formed. The valve member is displaceably arranged inside the receiving space of the valve sleeve between a closed position and an open position and opens in the outlet. The valve member has at least one valve member wall which is opposite the at least one inlet opening and in which a through-opening which is opposite the corresponding inlet opening in the open position is formed. The valve member forms a hollow space in which a diffuser insert which redirects the hydraulic fluid in the direction towards the outlet is arranged.

### FIELD OF THE INVENTION

The invention relates to a hydraulic actuator for orthopaedic devices having a control valve in a flow channel which has an inlet and an outlet for a hydraulic fluid. The control valve has a valve sleeve and a valve member, the valve sleeve has a sleeve wall which forms a receiving space and in which at least one radially orientated inlet opening is formed, the valve member is displaceably arranged inside the receiving space of the valve sleeve between a closed position and an open position, opens in the outlet and has at least one valve member wall which is opposite the at least one inlet opening and in which a throughflow opening which is opposite the corresponding inlet opening in the open position is formed.

### BACKGROUND

Orthopaedic devices are particularly but not exclusively prostheses, ortheses and exoskeletons. Prostheses replace appendages which are not or no longer present. Ortheses and exoskeletons are secured to appendages and support, influence or limit specific movements or positions of appendages relative to each other. In ortheses or exoskeletons which engage over joints, an upper portion is secured to a proximal appendage and a lower portion is secured to a distal appendage and they are pivotably connected to each other via a joint.

Prostheses can have a proximal upper portion and a distal lower portion, between which a joint device is arranged in order to achieve a pivotable connection between the upper portion and lower portion.

Actuators or resistance devices can be arranged between a proximal upper portion and a distal lower portion of an orthopaedic device and influence a pivoting movement between the upper portion and the lower portion. The influence can take place in terms of providing resistance, for example, via a hydraulic damper or a pneumatic damper. In order to actively support a movement, a drive or a power store is associated with the orthopaedic device in order to actively bring about a movement or in order to convert movement energy and to store it. Alternatively to joint devices, the orthopaedic devices can also influence a linear displacement of two components relative to each other.

In order to be able to influence the movement between two components, for example, an upper portion and a lower portion, of a joint device, devices for influencing the flow resistance are arranged in a flow channel of the hydraulic actuator which may be in the form of a hydraulic drive or passive damper. Often, one or more control valves via which the flow cross section can be adjusted are arranged in a flow channel between two hydraulic chambers which are separated by a piston. The adjustment can be carried out particularly during use of the orthopaedic device on the basis of sensors. To this end, sensor data are acquired and transmitted to a control device. In the control device, the sensor data are evaluated during use of the orthopaedic device and converted into actuation signals, as a result of which the control valve is adjusted. It is therefore possible to adapt the orthopaedic device to the respective situation for use.

As a result of the comparatively small dimensions within orthopaedic devices and the high pressures applied therein, high flow speeds are reached within the flow channels and therefore also within the control valves. This results in sometimes high noise loads which are perceptible both for the user of the orthopaedic device and for people in the surrounding area. Furthermore, hydraulic actuators in the areas of the control valves are prone to flow losses, which results in an increased basic resistance within the orthopaedic device. The precision of the control is thereby made worse and the pivoting behaviour of the joint components or the movement behaviour of the components between which the hydraulic actuator is arranged is permanently influenced.

### SUMMARY

An object of the present invention is to provide a hydraulic actuator for an orthopaedic device and a control valve with which a more comfortable use and improved control behaviour can be provided.

This object is achieved with a hydraulic actuator. Advantageous embodiments and developments of the invention are disclosed in the description and the figures.

The hydraulic actuator for orthopaedic devices having a control valve in a flow channel which has an inlet and an outlet for hydraulic fluid, wherein the control valve has a valve sleeve and a valve member and the valve sleeve has a sleeve wall which forms a receiving space and in which at least one radially orientated inlet opening is formed, wherein the valve member is displaceably arranged inside the receiving space of the valve sleeve between a closed position and an open position and opens in the outlet, wherein the valve member has at least one valve member wall which is opposite the at least one inlet opening and in which a throughflow opening which is opposite the corresponding inlet opening in the open position is formed, is characterized in that the valve member forms a hollow space in which a diffuser insert which redirects the hydraulic fluid in the direction towards the outlet is arranged. The diffuser insert prevents the radial influx of hydraulic fluid from striking the opposite wall of the valve member or mixing with an incoming hydraulic flow from another inlet opening or striking it. This would result in turbulent flows within the hollow space within the valve member and therefore in high noise loads and an increased flow resistance, particularly in completely open throughflow openings. The diffuser insert within the hollow space of the valve member redirects the hydraulic fluid so that, instead of striking a valve member wall at right angles, it is redirected in the direction towards the outlet. The formation of dead spaces and turbulence within the hollow space of the valve member is thereby avoided so that the flow resistance and the noise generation are reduced.

In one embodiment, the hollow space is configured in a closed manner except for the at least one through-opening and an outlet opening because in particular the end wall, which is opposite the outlet opening, of the hollow space is closed. Within the valve member wall, a plurality of throughflow openings corresponding to inlet openings in the sleeve wall can be configured in order to be able to introduce a plurality of partial flows of the hydraulic fluid into the hollow space. The otherwise closed configuration of the hollow space promotes the effective redirection of the hydraulic fluid.

In one embodiment, the diffuser insert is produced separately from the valve member and secured on or in the valve member. It is thereby possible to produce the diffuser insert from a different material from that of the valve member, in particular from a different material from that of the valve member wall, in which the inlet opening or the inlet openings is/are formed. The separate production of the diffuser insert further allows adaptation of the external contour or the geometry of the diffuser insert to different conditions of use or models and different hydraulic actuators. Furthermore, the production of complex three-dimensional structures with a free capacity for access from outside is simpler than within a sleeve-like valve member. Alternatively, the valve member is an integral component of the valve member and, for example, produced in the context of an additive production method, in which complex three-dimensional structures can be produced during the initial forming method as a result of a layered structure of material layers.

In one embodiment, the valve member has a plurality of through-openings, the diffuser insert has partition walls which are arranged between the through-openings. It is thereby possible to separate the flow channels of the respective through-openings and to redirect them individually. Different flow conditions can prevail inside flow channels so that the redirection of partial flows can also be brought about in different manners in order to achieve optimum influence and redirection of the partial flows.

In one embodiment, the partition walls do not extend as far as the outlet but instead subdivide the hollow space of the valve member only over a partial quantity in an axial extent so that the individual partial flows of the hydraulic fluid are combined even within the hollow space. In this case, it is advantageous if the partition walls taper in the direction towards the outlet so that a gradual expansion of the cross section for the fluid flow is produced in order to maintain or generate a flow which is as weakly turbulent as possible, in particular in order to maintain or generate a laminar flow. In an alternative, the partition walls extend as far as the outlet so that the individual partial flows are combined only after the outlet.

In one embodiment, the valve member is rotatably or displaceably supported in the valve sleeve. As a result of the rotation or displacement of the valve member in the valve sleeve, different cross sections for the hydraulic fluid in the hollow space of the valve member are provided. The generally present inlet cross section results from the overlapping geometries in the inlet opening with the opposite through-opening. In a static valve sleeve and a valve member which is movably supported relative thereto, the inlet cross section can be increased or decreased by the relative movement and, in an extreme case, completely closed. Via a corresponding geometry both of the inlet opening and of the through-opening, it is possible to generate for each rotation angle or for each displacement position an optimum cross section for the inflow opening. For example, it may be advantageous and desirable on the basis of a closed position to bring about initially only a slow increase of the inlet cross section and to allow the increase to increase more progressively only from a predetermined rotation angle or from a displacement position in order then to reduce the increase of the increase as far as a completely open position in order to allow a fine adaptation. Vice versa, the change per unit of the displacement path or per adjustment angle can also be selected to be small in a central opening region in order to allow a fine adjustment in a central damping region or activation region.

In one embodiment, the valve member is connected to an adjustment device with which it is possible to adjust or move the valve member into the desired position and to retain it in the desired position. The adjustment device can be connected to a manual or motorized drive, the adjustment device can also be connected to a mechanical drive, for example, in order to bring about opening or closing of the inlet cross section in a manner dependent on the path or loading.

In one embodiment, the diffuser insert is configured in a tapering manner in the direction towards the outlet in order to slowly allow an increase of the flow cross section in the direction towards the outlet.

The valve sleeve can be configured as part of a housing of the hydraulic actuator, for example, part of a damper housing. Alternatively, the valve sleeve is in the form of a separate component so that a control valve comprising a valve sleeve, valve member and diffuser insert can be in the form of a modular unit and produced separately. Such a control valve can then be inserted into the hydraulic actuator and where applicable connected to the adjustment device.

The inlet and outlet can be connected in technical flow terms to a piston/cylinder unit of the orthopaedic device and be connected to two chambers which are separated by the piston of the piston/cylinder unit. A hydraulic circuit between the inlet and the outlet is thereby produced. In principle, it is possible for only one flow channel to be present between the chambers of the piston/cylinder unit, in which the control valve is arranged. The diffuser insert is then active in only one direction and in the opposite direction the inlet would then be the outlet and the hydraulic fluid would flow at a high pressure through the hollow space and the through-openings back into the inlet opening. Preferably, a plurality of control valves are arranged in the hydraulic connections between the chambers, in particular in order to allow a separate influence of the adjustment movements.

In one embodiment, the inlet opening and/or the through-opening are orientated obliquely relative to the longitudinal extent of the control valve and are in the form of a slot. The slot can be configured in a rectilinear manner, that is to say, with a constant gradient, at the circumference of the valve sleeve and/or the valve member. Alternatively, the slot is curved or at least partially curved, whereby different overlaps of the inlet opening and through-opening can be obtained with a uniform adjustment movement. The width of the slot can change along the longitudinal extent thereof in order to obtain an increase or decrease of the width along the longitudinal extent thereof or along the axial extent of the control valve. It is thereby possible to achieve an adapted change of the inlet cross section in the hollow space.

The invention also relates to an above-described control valve per se, which is in the form of a modular unit and is formed from a valve sleeve and a valve member with a corresponding diffuser insert.

### DESCRIPTION OF THE DRAWINGS

Exemplary embodiments of the invention will be more particularly elucidated below with reference to the appended figures, In the figures:

FIG. 1 shows a schematic illustration of an applied prosthesis;

FIG. 2 shows a schematic illustration of a prosthetic leg;

FIG. 3 shows a sectioned illustration of a control valve;

FIG. 4 shows an individual illustration of a diffuser insert;

FIG. 5 shows a sectioned illustration of a control valve arrangement;

FIG. 6 shows a perspective illustration of a diffuser insert;

FIG. 7 shows a view of the diffuser insert obliquely from the rear;

FIG. 8 shows a sectioned illustration through a valve member with a diffuser insert;

FIG. 9 shows an exploded view of a control valve;

FIG. 10 shows the control valve according to FIG. 9 in the open position;

FIG. 11 shows a perspective illustration of the control valve according to FIG. 10;

FIG. 12 shows a longitudinally sectioned illustration through a control valve;

FIG. 13 shows a cross sectioned illustration through an open control valve;

FIG. 14 shows a perspective illustration of a closed control valve;

FIG. 15 shows sectioned illustrations through the control valve according to FIG. 14;

FIG. 16 shows a perspective illustration of a control valve;

FIG. 17 shows a partially transparent illustration of the control valve according to FIG. 16;

FIG. 18 shows a partially closed illustration of the control valve according to FIG. 17;

FIG. 19 shows an illustration of the flow path in the control valve according to FIG. 18; and

FIG. 20 shows a sectioned illustration of the control valve according to FIG. 16.

### DESCRIPTION

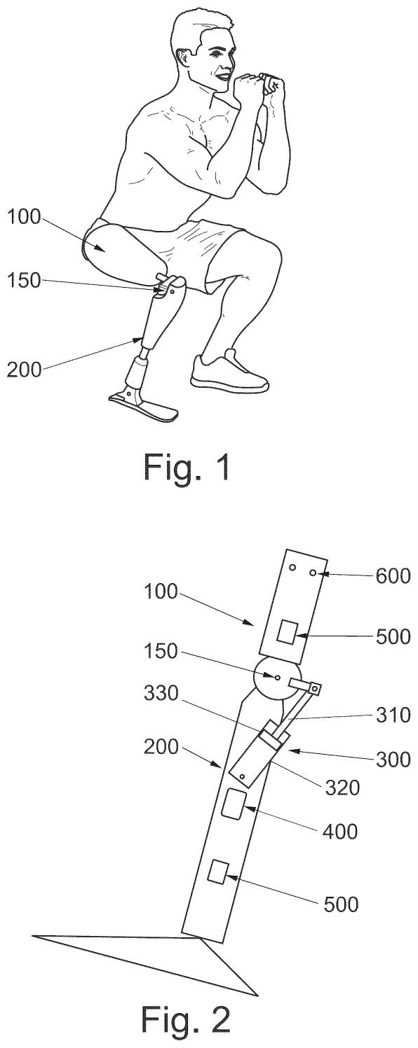

FIG. 1 shows a person using an orthopaedic joint device of a lower extremity with an upper portion 100 which has a prosthesis shaft and a lower portion 200 in the form of a lower leg portion having a prosthetic foot which is distally secured thereto. The upper portion 100 is pivotable relative to the lower portion 200 about a pivot axis 150.

FIG. 2 shows a schematic illustration of an orthopaedic joint device in the form of a prosthesis of the lower extremity. The orthopaedic joint device has an upper portion 100 and a lower portion 200 which are pivotably supported on each other about a pivot axis 150. In order to influence the relative pivoting of the upper portion 100 relative to the lower portion 200 about the pivot axis 150, there is arranged on the upper portion 100 and on the lower portion 200 an actuator 300 which is in the form of a hydraulic damper in the exemplary embodiment illustrated. The hydraulic damper 300 is in the form of a piston/cylinder unit and has in a housing a cylinder which is subdivided into two hydraulic chambers 310, 320 by a piston 330 which is connected to a piston rod. At least one technical flow connection is formed between the two hydraulic chambers 310, 320 so that hydraulic fluid can flow into one chamber when the chamber is increased and, vice versa, can flow out of a chamber when the chamber is decreased, and can flow into the opposite chamber. In the technical flow connection, which is not illustrated, between the two chambers 310, 320, a control valve which is described below in greater detail is arranged. In alternative embodiments, the actuator 300 may be in the form of an active actuator having a drive, for example, an electric motor, a power store or another type of drive in order to influence a pivot movement of the upper portion 100 relative to the lower portion 200. In the exemplary embodiment illustrated, the actuator 300 is in the form of a passive actuator and provides resistance against a flexion movement and where applicable an extension movement. The actuator 300 is connected to a control device 400 which is arranged on the lower portion 200 in the exemplary embodiment illustrated. The control device 400 is connected to sensors 500 which are arranged on the upper portion 100 and/or the lower portion 200. The sensors 500 detect state variables of the joint device, for example, positions, locations, forces, moments, accelerations or positions of components in space or relative to each other, wherein a large number of sensors can be used in order to detect the desired state variables of the orthopaedic joint device. The sensors 500 are connected to the control device 400 and transmit corresponding sensor data or sensor signals to the control device 400, wherein on the basis of the sensor values or sensor data the actuator 300 is changed with respect to the capacity thereof to influence the pivot movement or pivotability of the upper portion 100 relative to the lower portion 200. For example, the flexion resistance and/or extension resistance is changed in accordance with the sensor values. To this end, valves or throttles are adjusted. In alternative embodiments, magnetic fields can be changed in order to change magnetorheological fluids with respect to their viscosity. In a mechanical brake device, brake forces can be increased or decreased in order to generate an adapted resistance behaviour of the actuator 300. In an active drive, which has at least one electromechanical actuator, resistances against a movement can be applied via currents and voltages or movements can also be supported. Besides applying moments and moment paths, trajectories can be followed via control algorithms and sensor information items concerning the pivot movement of the upper portion 100 and lower portion 200 or system properties in the context of an impedance or admittance control can be emulated. For example, the behaviour of a linear or non-linear spring, the behaviour of a damper or an inertia can be emulated, or a combination of a plurality of properties, whereby a pivot movement counter to a flexion can be influenced. Such a control affords a high level of flexibility. As a result of such control operations, pivot movements can also be actively supported. If a moment or a force is applied via an actuator without pivoting taking place, for example, because external and internal forces are in equilibrium, the pivot movement is also counteracted with a resistance. Via actuators, energy stores can also be activated and deactivated, for example, a hydraulic resilient store, transmissions of drives can be changed and/or they can be connected and disconnected. These types of actuation can also be used to influence the pivot movement. Resistances are forces and moments which are applied by actuators in order to influence a pivot movement. A resistance can act counter to a pivot movement but also be a support of a pivot movement, that is to say, can act in the direction of the pivoting.

Biosignal sensors 600 which are configured to detect muscle activities or control of a muscle or the muscle system and which generate biosignals which are transmitted to the control device 400 are formed on or secured to the upper portion 100, for example, on a prosthesis socket or a retention member. The biosignal sensors 600 which may be in the form of deflector electrodes are connected to the control device 400 via a conductive connection or wirelessly and transmit corresponding signals to the control device 400 which also activates, deactivates or modulates the actuator 300 on the basis of the biosignals in order to change the resistance or to influence the movement behaviour or the displaceability of the components relative to each other.

Unlike the configuration of the orthopaedic joint device as a prosthesis, it can also be in the form of an orthesis, wherein in place of the lower leg portion as a lower portion 200 a lower leg rail is formed on an artificial knee joint. The upper portion 100 is then an upper leg rail which can be secured to the upper leg via corresponding fixing devices, such as belts, bowls or sleeves. The biosignal sensors 600 are fixed either separately to the upper leg or another muscle system group and can also be arranged on the fixing device. The orthopaedic joint device can also be in the form of an exoskeleton as a special type of orthesis. Alternatively to the embodiment illustrated as an orthopaedic joint device of the lower extremity, the orthopaedic joint device can also be configured for an upper extremity, for example, in the form of an artificial shoulder joint or artificial elbow joint for a prosthetic arm or as an orthesis or an exoskeleton for an arm.

The activation, deactivation and where applicable modulation of the actuator for influencing a pivot movement or the pivotability of the joint device is carried out on the basis of at least one signal from a sensor in such a manner that the influence of the pivot movement or pivotability counter to a flexion is increased if a corresponding sensor signal is present. The term “influencing the pivot movement” is also intended to be understood to mean that a pivot movement is blocked so that the lower portion 200 is locked relative to the upper portion 100 and no relative movement takes place and can take place between the two portions. The influence of the pivotability of the upper portion 100 relative to the lower portion 200 can therefore be carried out as a blocking of the joint in the flexion direction or an increase of the flexion resistance. The influence is carried out either by a passive actuator or by an active actuator. In order to reduce the increased flexion resistance of the joint device again, this is carried out on the basis of sensor values, that is to say, values which are transmitted from the sensor 500 or the sensors 500 and/or biosignal sensors 600 to the control device 400. Such a control of the orthopaedic joint device, for example, a lower extremity, is, for example, advantageous and expedient for a standing function, in which a flexion, for example, of the knee joint, is intended to be made more difficult or blocked. The influence of the pivotability is carried out, for example, via an adjustment of a control valve.

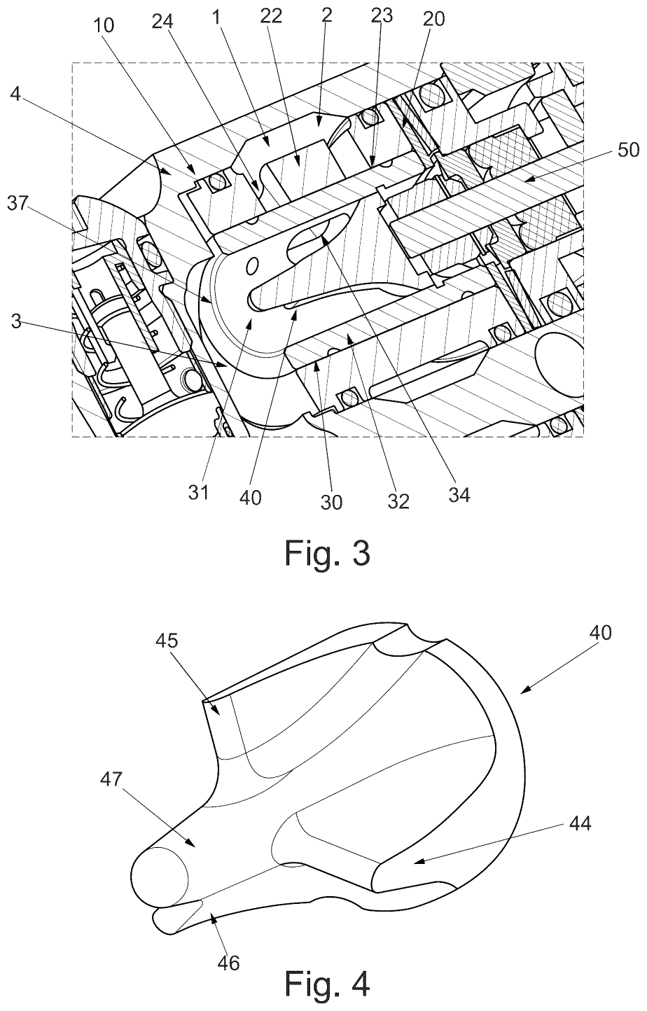

FIG. 3 illustrates a sectioned view of a control valve 10 which is arranged in a flow channel 1 which is located between one of the two chambers 310, 320 which are not illustrated. Hydraulic fluid is transported to an inlet 2 of the control valve 10 inside the flow channel. As soon as the hydraulic fluid has flowed through the control valve 10, it leaves it through an outlet 3. The control valve 10 has a valve sleeve 20 which is configured in a substantially cylindrical manner in the exemplary embodiment illustrated. The valve sleeve 20 has a sleeve wall 22 which surrounds a receiving space 23. A valve member 30 is arranged inside the receiving space 23. Hydraulic fluid is directed from the inlet 2 from the flow channel 1 through inlet openings 24 in the direction of the valve member 30. The sectioned illustration of FIG. 3 illustrates only one inlet opening 24 which is subdivided into two partial inlet openings. The inlet opening 24 is orientated radially so that hydraulic fluid which is transported from the exterior through the flow channel 1 which circumferentially surrounds the valve sleeve 20 to the inlet opening 24 can reach the interior of the valve sleeve 20. The flow channel 1 is arranged annularly around the valve sleeve 20 so that the hydraulic fluid can be directed in the direction towards the valve member 30 by a plurality of inlet openings.

The valve member 30 is displaceably supported within the receiving space 23 of the valve sleeve 20, rotatably supported in the exemplary embodiment illustrated. As a result of the rotation of the valve member 30 within the valve sleeve 20, it is possible to open and close the control valve 10. To this end, the valve member 30 is rotated between a closed position and an open position. In the intermediate positions, the control valve 10 is partially open or partially closed so that a reduced volume flow of the hydraulic fluid is produced. An increased flow resistance is accordingly present in the hydraulic system so that the resistance of the hydraulic actuator can be adjusted. In order to allow the hydraulic fluid to flow from the inlet 2 to the outlet 3 through the inlet opening 24, the valve member 30 has at least one valve member wall 32 which is opposite the inlet opening 24 or inlet openings and which is formed in the at least one through-opening 34 which is opposite the corresponding or opposite inlet opening 24 in an open position. In the open position, as a result of the superimposition of the inlet opening 24 and the through-opening 34, a maximum cross section for the throughflow opening for the hydraulic fluid is produced. In the closed position, the cross section of the throughflow opening is zero so that no hydraulic fluid can flow from the inlet 2 to the outlet 3. By rotating the valve member 30 within the receiving space 23, different throughflow cross sections are produced in order to adjust the desired hydraulic resistance.

Alternatively to a rotation which is illustrated in FIG. 3, the change of the throughflow cross section can also be carried out by a displacement of the valve member 30 relative to the valve sleeve 20. The rotation of the valve member 30 is carried out about a rotation axis which also defines the longitudinal extent of the control valve 10, during a displacement of the valve member 30 within the valve sleeve 20 the displacement direction is the longitudinal extent of the control valve 10.

In order to avoid a leakage, two sealing rings 25 which bring about a sealing with respect to a housing or the hydraulic line at both sides of the flow channel 20 are arranged on the outer circumference of the valve sleeve 20. In order to rotate or displace the valve member 30 relative to the valve sleeve 20, there is arranged on the valve member 30 an adjustment device 50, via which the valve member can be rotated. In the exemplary embodiment illustrated, the adjustment device 50 is in the form of a shaft which is connected to a drive or a manually actuatable adjustment element or adjustment wheel. The adjustment can be carried out manually or via a motorized drive which is preferably controlled on the basis of sensors via a control device in order to bring about an adaptation of the hydraulic resistance on the basis of sensor data.

There is formed inside the valve member 30 a hollow space 31 which is formed cylindrically in the exemplary embodiment illustrated. There is arranged inside the hollow space 31 a diffuser insert 40 which is arranged in a rotationally secure manner and in a non-displaceable manner in the longitudinal extent of the valve member 30 within the hollow space 31. The end, which is opposite the outlet 3, of the valve member 30 is sealed, in particular sealed by the diffuser insert 40. To this end, the diffuser insert 40 has a substantially cylindrical end piece which is fitted inside the hollow space 31. The cylindrical end piece reaches virtually as far as the through-openings 34 which in the exemplary embodiment illustrated are located on the same circumferential region which is located between the sealing rings as are the inlet openings 24. In the exemplary embodiment illustrated, the diffuser insert 40 is in the form of a separate component which is inserted and secured inside the valve member 30. Alternatively to a multi-piece configuration, the diffuser insert 40 can also be configured integrally with the valve member 30, either produced by an additive production method or by a corresponding separation method from a solid material.

The diffuser insert 40 tapers from the rear end thereof which closes the hollow space 31 in the direction towards the outlet 30 and has a rounded shape so that hydraulic fluid which flows radially from the outer side through the inlet opening 24 and the through-opening 34 is redirected by the diffuser insert by 90° in the direction towards an outlet opening 37 of the hollow space 31. As a result of the change, which is produced in the direction towards the outlet opening 37, of the shape of the surface of the diffuser insert 31, the flow cross section of the region of the hollow space 31 which is formed between the inner wall of the valve member 30 and the diffuser insert 40 is increased. As a result of the rounded surface shape with the curvature in the direction towards the outlet opening 37 and the gradual increase of the flow cross section, a redirection of the flow direction and a deceleration of the flow speed are carried out so that occurrences of turbulence within the hydraulic fluid are avoided or at least reduced.

The control valve 10 has a plurality of through-openings 34 and inlet openings 24 which are arranged radially around the valve sleeve 20 and the valve member 30, preferably with identical spacing. It is thereby possible to allow in total a comparatively high throughflow of the hydraulic fluid to flow through a large number of through-openings 34 into the hollow space 31. In such a configuration with a plurality of inlet openings 24 and through-openings 34, it is advantageous to arrange in the region of the through-openings 34 partition walls 44, 45, 46 which are illustrated in FIG. 4 in the perspective illustration of the diffuser insert 40. The diffuser insert 40 in FIG. 4 is an integrally formed moulded member which has three partition walls 44, 45, 46 which reach in the fitted state within the valve member 30 as far as the inner wall of the hollow space 31. In a cylindrical configuration of the hollow space 31, the partition walls 44, 45, 46 extend rectilinearly from the rear end of the diffuser insert 40 in the direction towards the outlet 3 or the outlet opening 37. Cavity-like, rounded flow faces which extend from the external circumference of the valve member 40 radially inwardly in the direction towards the central member 47 are formed between the rear walls 44, 45, 46, respectively. The central member 47 is formed conically and tapers in the direction towards the outlet opening 37. The central member 47 advantageously terminates in front of the outlet opening 37 in order to provide the entire cross section of the hollow space 31 in the region of the outlet opening 37 as a flow cross section for the hydraulic fluid. The partition walls 44, 45, 46 terminate in front of the tip of the central member 47, wherein a rounded transition both between the cavities between the partition walls 44, 45, 46 and radially inwardly is produced in order to avoid occurrences of turbulence within the hydraulic fluid.

At the rear end of the diffuser insert 40, there is formed a recess in the form of a rotation prevention member which can engage in a positive-locking manner in a projection inside the valve member 30.

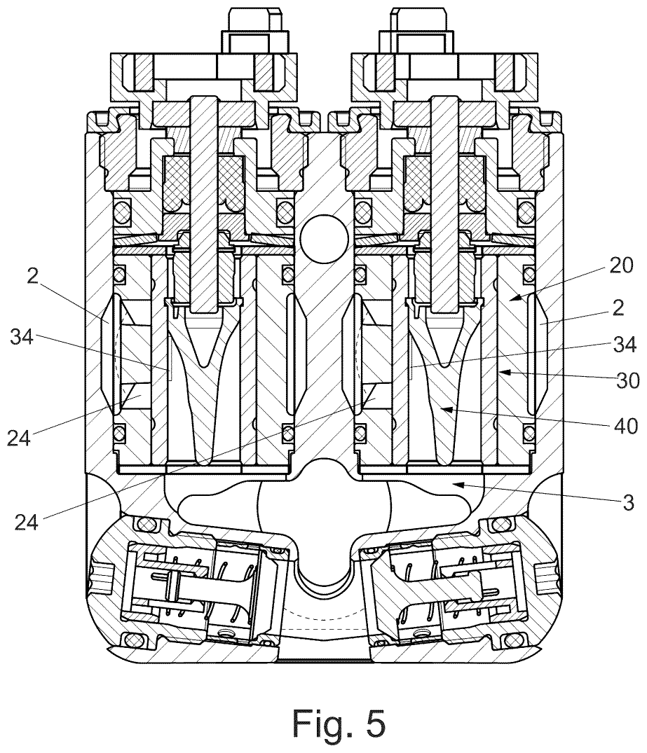

FIG. 5 illustrates a cross sectioned illustration of two control valves 10 which are arranged parallel with each other and which open in a common outlet 3. The hydraulic fluid can be transported from the outlet 3 to the respective chamber of the hydraulic actuator by check valves. In principle, it is also possible to permit a transposed movement of the hydraulic fluid without check valves.

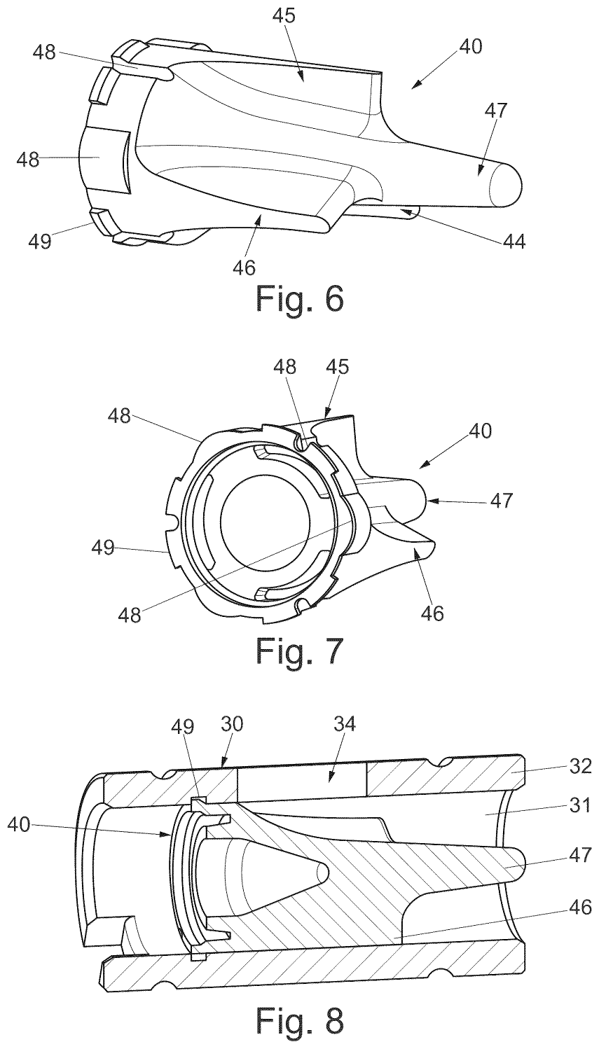

FIG. 6 illustrates a variant of the diffuser insert 40. This diffuser insert 40 also has three partition walls 44, 45, 46 which are orientated in an identically distributed manner over the circumference relative to each other so that the centre planes of the partition walls 44, 45, 46 are orientated at an angle of 120° relative to each other. There are formed at the rear end of the diffuser insert 40 rotation prevention members 48 and axial securing members 49 which prevent displacement and rotation of the diffuser insert 40 inside the valve member 30.

FIG. 7 illustrates the diffuser insert 40 according to FIG. 6 as a rear view. The rotation prevention members 48 and the axial securing members 49 at the rear end are visible, as are the configuration as a hollow member and the flow surface which extends forwards in the direction towards the tip of the central member 47 in a curvature so that the diffuser insert 40 tapers in the flow direction from the inlet 2 to the outlet 3. The partition walls 44, 45, 46 also taper in the direction towards the tip of the central member 47, preferably with a curved contour.

FIG. 8 shows a cross sectioned view of the valve member 30 with the inserted diffuser insert 40 according to FIGS. 6 and 7. The external circumference of the partition walls abuts the inner wall of the valve member 30 in this case and the abutment of the partition wall 46 against the inner wall of the hollow space 31 is illustrated. The through-opening 34 inside the valve member wall 32 is arranged between the two other partition walls so that three inflow regions which are separated from each other are formed within the hollow space 31 by the partition walls 44, 45, 46. The hydraulic fluid flow, which flows in radially through the through-openings 34 from outside, is redirected at the surface of the diffuser insert 40 through 90° and directed along the tapering central member 47 in the direction towards the outlet opening.

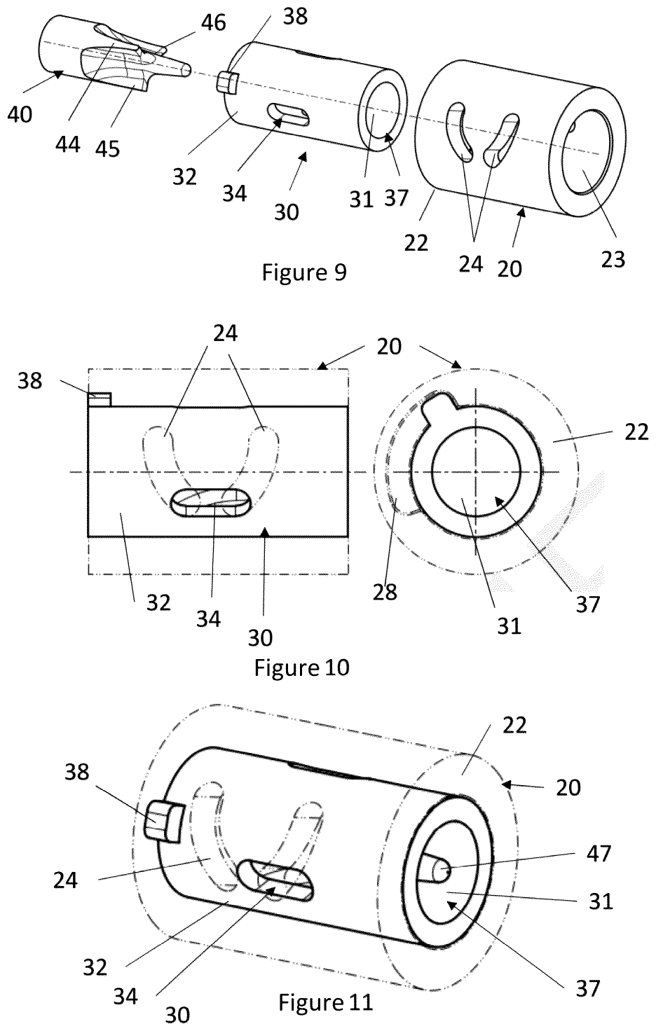

FIG. 9 illustrates an exploded view of the control valve with the three main components valve sleeve 20, valve member 30 and diffuser insert 40. The valve sleeve 20 is configured in a cylindrical manner and has a substantially closed sleeve wall 22 in which inlet openings 24 are uniformly arranged in a manner offset by 120° around the circumference. In the exemplary embodiment illustrated, the inlet openings 24 are formed by two partial openings which are in the form of curved slots.

The slots are orientated in a manner inclined relative to the longitudinal extent of the valve sleeve 20 or alternatively they may also be configured so as to be orientated rectilinearly and obliquely to the longitudinal extent. The receiving space 23 in the form of a cylindrical shell and receiving space for the valve member 30 is formed inside the valve sleeve 20. The inlet openings 24 extend through the sleeve wall 22 so that fluid can flow through the inlet opening 24 in the direction towards the receiving space 23 and to the valve member 30 through the sleeve wall 22.

In the embodiment illustrated, the valve member 30 also has a cylindrical external circumference. The valve member 30 is formed by the valve member wall 32 in the form of a sleeve-like insert 32, wherein three through-openings are arranged one behind the other in the circumferential direction in a state offset by 120° over the circumference in the sleeve wall 32. The through-openings, of which only one through-opening 34 can be seen, are in the exemplary embodiment illustrated in the form of a rectilinear slot which extends along the longitudinal extent of the valve member 30. The valve member 30 forms the hollow space 31 with the outlet opening 37. At the outer side of the valve member 30, a positive-locking element 38 in the form of a stop limitation is arranged at the rear end which is opposite the outlet opening 37 in order to limit a rotation within the valve sleeve 20.

The diffuser insert 40 is formed, similarly to the previously described diffuser inserts, with three partition walls 44, 45, 46 and is arranged in a rotationally secure and axially non-movable manner inside the hollow space 31 of the valve member 30. All the components of the control valve 10 are orientated coaxially and are arranged one in the other. Alternatively to an arrangement of the through-opening 34 as a rectilinear slot along the longitudinal extent of the valve member 30, it can also be configured in an inclined manner relative to the longitudinal extent at the circumference of the valve member wall 33. The configuration may be formed similarly to the through-openings 34 in the sleeve wall 22 of the valve sleeve 20.

Similarly, the inlet opening 24 can be in the form of a longitudinally orientated slot. It is also possible for both openings, both the inlet opening 24 and the through-opening 34, to be configured similarly to the inlet opening 24 which is illustrated in FIG. 9, wherein the dimensions, shapings and orientations have to be configured so that no overlaps at all of the openings are present for completely closing the control valve. In order to move into the open position, the valve member 30 is rotated relative to the valve sleeve 20 until a maximum coverage of the openings is present.

FIG. 10 and FIG. 11 illustrate the control valve in the open position. FIG. 10 shows a longitudinal section and a cross section, wherein the valve sleeve 20 is illustrated with broken lines. FIG. 11 shows a perspective view with a maximum throughflow opening which is obtained by the coverage of the inlet opening 24 over the entire width with the through-opening 34. Within the valve sleeve 20, a guide channel 28 in which the positive-locking element 38 of the valve member 30 is guided is formed. FIG. 10 illustrates, in the right-hand illustration, the maximum open position with the stop of the positive-locking element 38 at the end of the guide 28. FIG. 11 shows that the front end of the central member 47 terminates shortly before the end of the outlet opening 37 of the hollow space 31 of the valve member 30. The hydraulic fluid which flows in radially from the exterior is redirected by the coverage of the inlet opening 24 with the through-opening 34 in the direction towards the diffuser insert 40, wherein the hydraulic fluid strikes the curved surface between the partition walls 44, 45 and is then redirected in the direction towards the outlet 37. A direct meeting of the hydraulic fluid partial flows, which flow in through the through-openings 34, is achieved by the complete hydraulic separation by the partition walls 44, 45, 46, wherein the partition walls 44, 45, 46 preferably extend as far as the end of the through-openings 34.

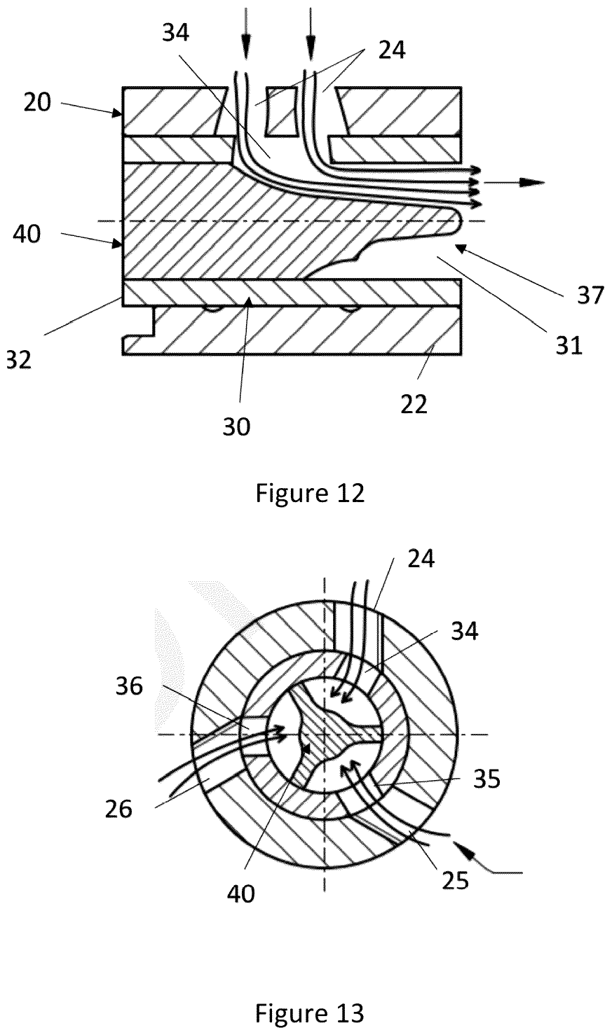

FIG. 12 illustrates a longitudinally sectioned view of the control valve 10 with the flow path present. The hydraulic fluid flows from the radially outer side through the inlet openings 24 and the through-openings 34 into the hollow space 31 and is redirected at the surface of the diffuser insert 40 and redirected by 90° in the direction towards the outlet 37.

FIG. 13 shows a cross sectioned view of the control valve 10 according to FIG. 9 in the mounted state. The three inlet openings 24, 25, 26 which are again connected to the flow channel or the inlet in technical flow terms are distributed uniformly over the circumference. Hydraulic fluid flows through the inlet openings 24, 25, 26 and the through-openings 34, 35, 36, which are orientated so as to correspond thereto, of the valve member 30 into the hollow space 31. The partial flows of the hydraulic fluid strike the valve member 40 and are prevented by the partition walls, in the region of the inflow, from directly meeting each other. The channels which are formed by the partition walls are combined in the direction towards the outlet 37 so that a common fluid flow of the hydraulic fluid bears on the outlet 37.

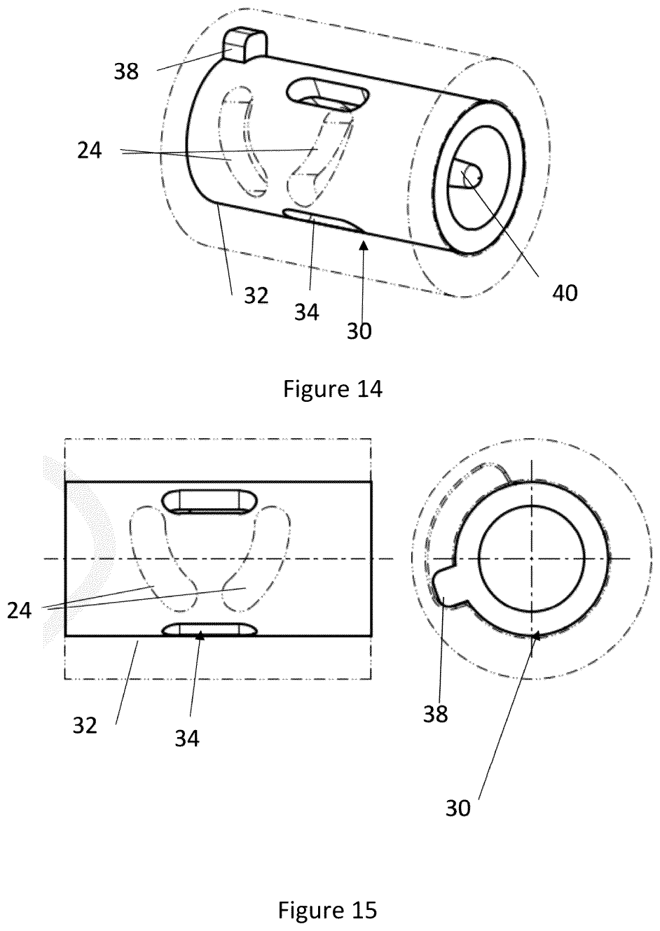

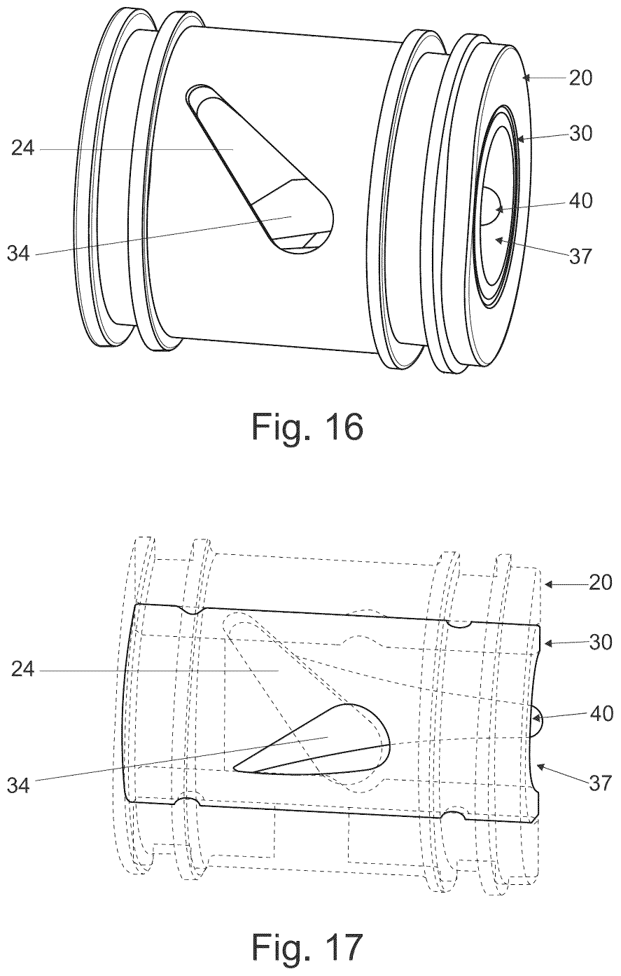

FIGS. 14 and 15 show the control valve in a closed position. The structure substantially corresponds to that of FIGS. 11 and 10. FIG. 15 shows that the positive-locking element 38 is rotated inside the guide in the valve sleeve up to the opposite stop. The valve member 30 is thereby moved from the open position into the illustrated closed position, in which no coverage at all of the inlet opening 24 with the through-openings 34 is present. Hydraulic fluid cannot flow through the inlet opening 24 from the exterior and through the through-openings 34, except for a potentially present leak which can be minimized or prevented by minimizing the gap widths.

FIG. 16 illustrates a variant of the control valve 10, in this instance a valve sleeve 20 is also illustrated with a valve member 30 arranged rotatably therein and a diffuser insert 40 which is arranged in a rotationally secure manner therein and which is axially secured. The differences from the preceding embodiments relate to the geometry and configuration of the inlet openings 24 and the through-openings 34. Three inlet openings and three through-openings are also provided here, alternative configurations with only two inlet openings and through-openings or more than three inlet openings and through-openings and where applicable with only one inlet opening and one through-opening are also possible.

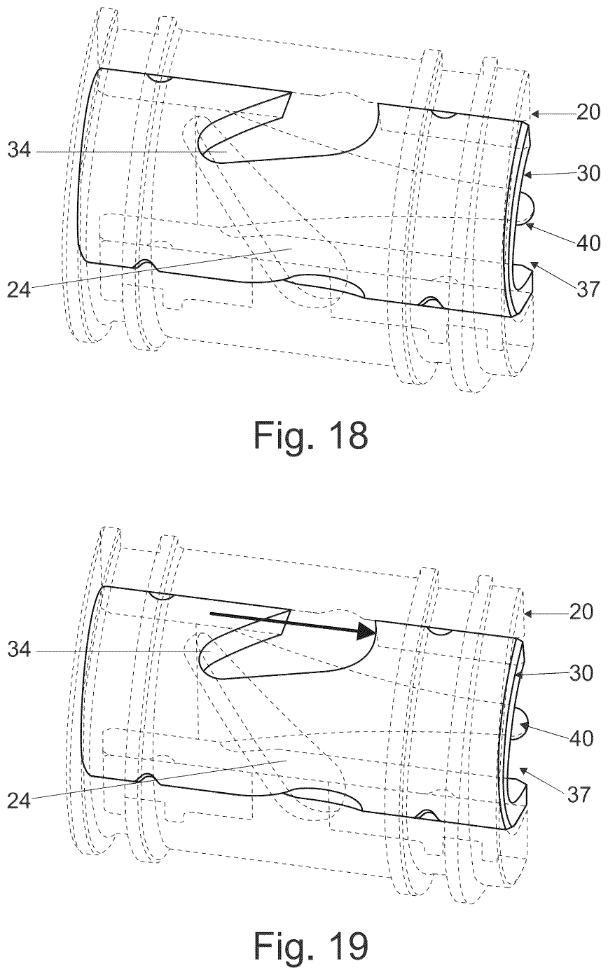

In the exemplary embodiment illustrated, the inlet openings 24 are in the form of slots which are configured in a manner inclined relative to the longitudinal extent of the control valve within the wall of the valve member 30 and the valve sleeve 20. The inclination of the inlet openings 24 and the inclination of the through-openings 34 are opposite so that the slots intersect each other in the course of a rotation from the illustrated open position to the closed position illustrated in FIGS. 18 and 19. In the embodiment illustrated, the width of the slots continuously increases from the rear end to the front end which faces the outlet 37 since the side flanks of the slots are not orientated parallel with each other but instead run apart from each other from the rear towards the front in the direction towards the outlet 37.

In FIGS. 18 and 19, the valve sleeve 20 and the valve insert 30 are located in a virtually closed position. The free throughflow cross section which results from the coverage of the inlet openings 24 and the through-openings 34 is reduced in comparison with the production according to FIG. 16. The position at which the hydraulic fluid is introduced into the hollow space 31 of the valve member 30 moves from approximately the centre of the valve member 30 to the rear end in the direction towards the shoulder of the diffuser insert which bears against the inner side of the hollow space 31 of the valve member 30. The arrow in FIG. 19 indicates the movement direction of the free throughflow cross section in the hollow space 31 when the valve member 30 is rotated downwards. The free throughflow cross section then moves from the rear position downwards and forwards. As a result of this displacement of the position of the free throughflow cross section forwards, the path of the hydraulic fluid from the throughflow openings to the surface of the diffuser insert 40 becomes greater so that an optimized redirection results. A reduced volume flow as a result of the smaller throughflow cross section, as illustrated in FIGS. 18 and 19, can be introduced into the hollow space nearer the surface of the diffuser insert 40 and be directed through in the channel between the partition walls in the direction towards the outlet 37.

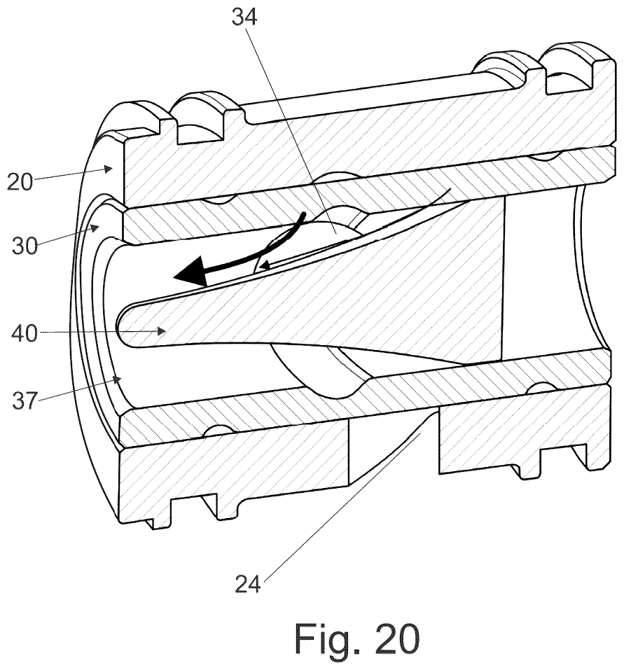

FIG. 20 shows the embodiment of FIGS. 16 to 19 as a cross sectioned illustration. The inlet opening in the valve sleeve 20 is not shown, the through-opening 34 moves relative to the inlet opening in the case of a corresponding rotation, wherein the free throughflow cross section of the superimposed inlet openings and through-openings moves both in the circumferential direction and in the axial extent. In the position according to FIGS. 18 and 19, the hydraulic fluid is introduced further into the hollow space 31 at the rear end, which is indicated by the longer, rear arrow. In the open position, according to FIGS. 16 and 17, a greater volume flow enters through the free throughflow cross section of the inlet openings and through-openings, which is indicated by the front, thicker arrow.

By adjusting or rotating the superimposition of the inlet opening and through-opening, the free throughflow cross section moves as therefore does the inflow of the hydraulic fluid into the hollow space in the direction towards the diffuser insert 40 along the longitudinal extent. In accordance with the relative position of the valve sleeve 20 and valve member 30, the axial position of the free throughflow cross section and the position of the incoming hydraulic fluid change. In this case, the diffuser insert 40 is formed so that an optimum situation in technical flow terms is achieved so that no flow or only a small turbulent flow is present during the inflow into the control valve.

1. A hydraulic actuator for orthopaedic devices, comprising:
a control valve in a flow channel which has an inlet and an outlet for a hydraulic fluid, wherein the control valve comprises
a valve sleeve and a valve member,
wherein the valve sleeve has a sleeve wall which forms a receiving space, wherein at least one radially orientated inlet opening is formed in the receiving space,
wherein the valve member is displaceably arranged inside the receiving space of the valve sleeve between a closed position and an open position, and wherein the valve member opens in the outlet of the flow channel,
wherein the valve member has at least one valve member wall which is opposite the at least one inlet opening, wherein the at least one valve member wall comprises at least one through-opening which is opposite the at least one inlet opening,
wherein the valve member forms a hollow space in which a diffuser insert is arranged, wherein the diffuser insert redirects the hydraulic fluid in a direction towards the outlet.

2. The hydraulic actuator as claimed in claim 1, wherein the hollow space is configured in a closed manner except for the at least one through-opening and an outlet opening.

3. The hydraulic actuator as claimed in claim 1 wherein the diffuser insert is separable from the valve member and is secured on or in the valve member, or wherein the diffuser insert is an integral component of the valve member.

4. The hydraulic actuator as claimed in claim 1 wherein the at least one through opening in the at least one valve member wall comprises a plurality of through-openings, and wherein the diffuser insert comprises partition walls are arranged between pairs of through-openings of the plurality of through-openings.

5. The hydraulic actuator as claimed in claim 4, wherein the partition walls extend as far as the outlet or terminate in front of the outlet.

6. The hydraulic actuator as claimed in claim 1, wherein the valve member is rotatably or displaceably supported in the valve sleeve.

7. The hydraulic actuator as claimed in claim 1 further comprising an adjustment device, and wherein the valve member is connected to the adjustment device.

8. The hydraulic actuator as claimed in claim 1 wherein the diffuser insert is configured in a tapering manner in the direction towards the outlet.

9. The hydraulic actuator as claimed in claim 1 wherein the valve sleeve is configured as part of a housing, or is a separate component from the housing.

10. The hydraulic actuator as claimed in claim 1 wherein the inlet and the outlet are connected in terms of flow to a piston/cylinder unit, and wherein the inlet and the outlet and are respectively connected to an inlet chamber and an outlet chamber which are separated by a piston.

11. The hydraulic actuator as claimed in claim 1 wherein the at least one inlet opening and/or the at least one through-opening is/are configured as a slot which is orientated obliquely relative to a longitudinal extent of the control valve.

12. The hydraulic actuator as claimed in claim 11, wherein the slot is configured in a rectilinear or curved manner and/or changes in terms of a slot width along a longitudinal extent of the slot.

13. A control valve as claimed in claim 1.
